(19) United States
(12) Patent Application Publication
(10) Pub. No.: US 2002/0035735 A1
(43) Pub. Date: Mar. 21, 2002

(54) Clonal propagation of primate offspring by embryo splitting

(76) Inventors: Gerald Schatten, Portland, OR (US); Anthony W.S. Chan, Aloha, OR (US)

Correspondence Address: KLARQUIST SPARKMAN, LLP, 121 SW SALMON STREET, SUITE 1600, PORTLAND, OR, 97204, US

(21) Appl. No.: 09/754,276

(22) Filed: Jan. 5, 2001

Related U.S. Application Data
(60) Provisional application No. 60/174,812, filed on Jan. 7, 2000.

(51) Int. Cl. A01K 67/027

(52) U.S. Cl. 800/14; 800/21

(57) ABSTRACT

The present invention relates to the clonal propagation of primate offspring by embryo splitting. Here, genetically identical nonhuman embryos may be produced as twin and larger sets by separation and reaggregation of blastomeres of cleavage-stage embryos. Furthermore, the present invention also relates to methods for producing embryonic stem cells and transgenic embryonic stem cells isolated from dissociated blastomeres.

### CROSS REFERENCE TO RELATED APPLICATIONS

The present invention is related to and claims the benefit of, under 35 U.S.C. § 119(e), U.S. provisional patent application Ser. No. 60/174,812, filed Jan. 7, 2000, which is expressly incorporated fully herein by reference.

### FIELD OF THE INVENTION

The present invention relates to methods for the clonal propagation of animals, specifically primates. The present invention also relates to methods for producing embryonic stem cells and transgenic embryonic stem cells.

### BACKGROUND OF THE INVENTION

The cloning of animals from adult somatic cells has lead to the creation of sheep (Wilmut et al., 385 NATURE 810-13 (1997)), cattle (Kato et al., 282 SCIENCE 2095-98 (1998)), mice (Wakayama et al., 394 NATURE 369-74 (1998)), and goats (Baguisi et al., 17 NATURE BIOTECH. 456-61 (1999)). Among the most compelling scientific rationales for cloning is the production of disease models. Cloned animals as models for disease show great promise because the genetics of each clone are invariable. Although the scientific rationales remain compelling, the death of clones as fetuses and newborns (Kato et al. (1998); Cibelli et al., 280 SCIENCE 1256-58 (1998); Hill et al., 51 THERIOGENOLOGY: 1451-65 (1999); Renard et al., 353 LANCET 1489-91 (1999); Wells et al., 10 REPROD. FERT. DEV. 369-78 (1998); and Wells et al., 60 BIOL. REPROD. 996-1005 (1999)) as well as reports of shortened telomeres (Shields et al., 399 NATURE 316-17 (1999)), which suggests that nuclear transfer does not reverse aging, imply some limitations to this cloning technique. Furthermore, mitochondrial heterogeneity in clones, due to the use of the different enucleated oocytes, also demonstrates that nuclear transfer results in genetic chimeras (Evans et al., 23 NATURE GENETICS 90-93 (1999)). Notwithstanding success in domestic species and rodents, similar breakthroughs in nonhuman primates have not followed (Wolf et al., 60 BIOL. REPROD. 199-204 (1999)).

Identical primates have immeasurable importance for molecular medicine, as well as implications for endangered species preservation and infertility. The lack of genetic variability among cloned animals results in a proportional increase in experimental accuracy, thereby reducing the numbers of animals needed to obtain statistically significant data, with perfect controls for drug, gene therapy, and vaccine trials, as well as diseases and disorders due to aging, environmental, or other influences. The “nature versus nurture” questions regarding the genetic versus environmental including maternal environment or epigenetic influences on health and behavior may also be answered. Consequently, genetically identical offspring, even with differing birth dates, may be investigated (e.g., in studies such as phenotypic analysis prior to animal production; serial transfer of germ line cells (e.g., the male germ cells) Brinster et al., 9 SEMIN. CELL DEV. BIOL. 401-09 (1998)), to address cellular aging beyond the life expectancy of the first offspring; and testing simultaneous retrospective (in the older twin) and prospective therapeutic protocols. Epigenetic investigations may be tested using identical embryos of the present invention implanted serially in the identical surrogate to demonstrate that, for example, low birth weight or other aspects of fetal development may have life long consequences (Leese et al., 13 HUM. REPROD. 184-202 (1998)), the decrease in the IQ of children is related to maternal hypothyroidism during pregnancy (Haddow et al., 341 N. ENGL. J. MED. 549-55 (1999)), or immunogenetics results in uterine rejection (Gerard et al., 23 NAT. GENET. 199-202 (1999); Clark et al., 41 AM. J. REPROD. IMMUNOL. 5-22 (1999); and Hiby et al., 53 TISSUE ANTIGENS 1-13 (1999)).

Cloning by embryo splitting promises advantages over nuclear transfer technology. Theoretically, but unfortunately not practically, nuclear transfer could have produced limitless identical offspring; however, genetic chimerism (Evans et al. (1999)), fetal and neonatal death rates (Kato et al. (1998); Cibelli et al. (1998); Hill et al. (1999); Renard et al. (1999); Wells et al. (1998); and Wells et al. (1999)), shortened telomeres (Shields et al. (1999)), and inconsistent success rates (Kato et al. (1998); Cibelli et al. (1998); Hill et al. (1999); Renard et al. (1999); Wells et al. (1998); and Wells et al. (1999)) preclude its immediate usefulness. These concerns notwithstanding, the contradictions and paradoxes raised by nuclear transfer have stimulated new studies on the molecular regulation of mammalian reproduction.

In contrast to nuclear transfer which result in genetic chimeras, offspring resulting from embryo splitting are expected to be fully identical (i.e., nuclear as well as cytoplasmic). The report from an infertility clinic on the high frequency of mitochondrial heteroplasmy after cytoplasmic therapy is worrisome. This unorthodox approach attempts to rescue aging oocytes retrieved from older women by the microinjection of cytoplasm from young donor oocytes. The combination of splitting and nuclear transfer, in which two triplets are produced by splitting and the third by nuclear transfer, may address the consequences of cytoplasmic inheritance.

Stem cell lines have been produced from human and monkey embryos (Shamblott et al., 95 PROC. NATL. ACAD. SCI. USA 13726-31 (1999) and Thomson et al., 282 SCIENCE 1145-47 (1999)). It is not yet known if stem cells from the fully outbred populations of humans or primates have the full totipotency of those from selected inbred mouse strains with invariable genetics.

This can now be evaluated within the context of the present invention, for example, by producing therapeutic stem cells from one multiple, later tested in its identical sibling, and in so doing, learning if stem cells might produce cancers like teratocarcinomas.

### SUMMARY OF THE INVENTION

The present invention is directed to methods for clonal propagation of an animal by embryo splitting. In a preferred embodiment, blastomeres are dissociated from an embryo. The blastomeres are then transferred to an empty zona, and cultured to an embryonic stage. Subsequently, the cultured embryos are then transferred to surrogate females, and a cloned animal is produced by parturition.

In another embodiment of the present invention, the animal may be a mammal, bird, reptile, amphibian, or fish. In another aspect of this method, the animal is a nonhuman primate, preferably a monkey.

In another embodiment of the present invention, the embryo is cultured to the 4- to 8-cell stage prior to transfer to the female surrogate. In another aspect of the invention the embryo is transgenic. In a further aspect of the invention, the embryos are frozen and stored prior to transfer to surrogate females. In a further aspect of the invention, the blastomeres are frozen and may serve as an embryonic stem cell repository.

In a preferred embodiment of the present invention, preimplantation genetic diagnosis is performed on an isolated blastomere from the embryo prior to transfer to the oviduct of a female surrogate. The methods used for this preimplantation genetic diagnosis include polymerase chain reaction (PCR), fluorescence in situ hybridization (FISH), single-strand conformational polymorphism (SSCP), restriction fragment length polymorphism (RFLP), primed in situ labeling (PRINS), comparative genomic hybridization (CGH), single cell gel electrophoresis (COMET) analysis, heteroduplex analysis, Southern analysis, and denatured gradient gel electrophoresis (DGGE) analysis.

The present invention is also directed to animals produced by the methods described herein. In a preferred embodiment, the animal is a primate. In another aspect of the present invention, the animal is a transgenic animal, preferably a transgenic primate.

Also within the scope of the present invention is the production of embryonic stem cells and transgenic embryonic stem cells from isolated blastomeres generated by the embryo splitting method. In a preferred embodiment, the split embryos are used to produce clonal offspring and the isolated blastomeres are used to produce an embryonic stem cell line. In a further embodiment, the split embryos are transgenic, and these split transgenic embryos are used to produce clonal transgenic offspring and the isolated transgenic blastomeres are used to produce transgenic embryonic stem cell lines.

The present invention also relates to methods of producing embryonic stem cells whereby blastomeres are dissociated from embryos and these cells are then cultured to produce stem cell lines. In a preferred embodiment, the methods described herein are used to produce primate embryonic stem cells. In another aspect of the invention, the methods described herein are used to produce transgenic embryonic stem cells, preferably transgenic primate embryonic stem cells.

The present invention is also directed to embryonic stem cells produced by the methods described herein. In a preferred embodiment, the embryonic stem cells are primate embryonic stem cells. In a further embodiment, the embryonic stem cells are transgenic, preferably transgenic primate embryonic stem cells.

The present invention also relates to methods for preimplantation genetic diagnosis of an embryo. In a preferred embodiment, blastomeres are dissociated from an embryo and genetic analysis is performed on a single blastomere. In a further embodiment of the present invention, the remaining blastomeres are cultured to an embryonic stage and subsequently implanted in a female surrogate. The methods used for the genetic analysis of the blastomere include PCR, FISH, SSCP, RFLP, PRINS, CGH, COMET analysis, heteroduplex analysis, Southern analysis, and DGGE analysis.

### DESCRIPTION OF FIGURES

FIGS. 1A-H: Embryo splitting and development of primates in vitro and after embryo transfer.

FIGS. 1A-B: A zona-free 8-cell stage rhesus embryo, fertilized in vitro, was dissociated into eight individual blastomere by mechanical disruption in Ca2⁺- and Mg2⁺-free medium.

FIGS. 1C-E: Two dissociated blastomeres were transferred into each of four empty zonae, thereby creating the four quadruplet embryos, each with two of the eight original cells. These embryos were cultured on a Buffalo Rat Liver cell monolayer. Multiple embryos were scored daily for development and structural normalcy.

FIG. 1F: Embryos showing signs of compaction were selected for transfer 1-3 days after splitting. Endocrine profiles were traced daily and implantation was confirmed by ultrasound on day 31 post transfer.

FIG. 1G: An abnormal quadruplet pregnancy in which the fetus was absent though the placenta appears normal.

FIG. 1H: The quadruplet pregnancy with normal fetal development that resulted in the birth of a normal female. Bar in A-F=120 μm; in G and H=5 cm.

FIG. 2: The allocation of embryonic cells to both the trophectoderm and inner cell mass cells was lower in multiple embryos versus controls. Controls had twice the cell number of the multiples at the blastocyst stage. Split rhesus embryos undergo compactation and blastocyst formation at similar chronological times as controls.

FIG. 3: Success rates of compaction and blastocysts. Developmental potential of reconstructed embryos decrease when advance stage embryos were split. Embryos split into twins display higher rates of compaction and blastocyst formation than embryos separated into triplets and higher orders.

FIG. 4: Developmental potential of each reconstructed embryo. Higher-order multiples displayed reduced developmental potential. The compaction rate was maintained even at a higher order of splitting, although a slight decrease was observed when three or more embryos were created. Unlike compaction, blastocyst formation rate was more sensitive to a higher order of splitting. The blastocyst rate was reduced by half when 3 embryos were created rather than 2, and development was arrested when splitting beyond sextuplets was attempted.

### DETAILED DESCRIPTION OF THE PRESENT INVENTION

Before the methods of the present invention are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

For convenience, the meaning of certain terms and phrases employed in the specification, examples, and appended claims are provided below.

The term “animal” includes all vertebrate animals such as mammals (e.g., rodents (e.g., mice and rats), primates (e.g., monkeys, apes, and humans), sheep, dogs, rabbits, cows, pigs), amphibians, reptiles, fish, and birds. It also includes an individual animal in all stages of development, including embryonic and fetal stages.

The term “primate” as used herein refers to any animal in the group of mammals, which includes, but is not limited to, monkeys, apes, and humans.

The term “totipotent” as used herein refers to a cell that gives rise to all of the cells in a developing cell mass, such as an embryo, fetus, and animal. In preferred embodiments, the term “totipotent” also refers to a cell that gives rise to all of the cells in an animal. A totipotent cell can give rise to all of the cells of a developing cell mass when it is utilized in a procedure for creating an embryo from one or more nuclear transfer steps. An animal may be an animal that functions ex utero. An animal can exist, for example, as a live born animal. Totipotent cells may also be used to generate incomplete animals such as those useful for organ harvesting, e.g., having genetic modifications to eliminate growth of a head, or other organ, such as by manipulation of a homeotic gene.

The term “totipotent” as used herein is to be distinguished from the term “pluripotent.” The latter term refers to a cell that differentiates into a sub-population of cells within a developing cell mass, but is a cell that may not give rise to all of the cells in that developing cell mass. Thus, the term “pluripotent” can refer to a cell that cannot give rise to all of the cells in a live born animal.

The term “totipotent” as used herein is also to be distinguished from the term “chimer” or “chimera.” The latter term refers to a developing cell mass that comprises a sub-group of cells harboring nuclear DNA with a significantly different nucleotide base sequence than the nuclear DNA of other cells in that cell mass. The developing cell mass can, for example, exist as an embryo, fetus, and/or animal.

The term “embryonic stem cell” as used herein includes pluripotent cells isolated from an embryo that are preferably maintained in in vitro cell culture. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells and their uses are well known to a person of skill in the art. See, e.g., U.S. Pat. No. 6,011,197 and WO 97/37009, entitled “Cultured Inner Cell Mass Cell-Lincs Derived from Ungulate Embryos,” Stice and Golueke, published Oct. 9, 1997, both of which are incorporated herein by reference in their entireties, including all figures, tables, and drawings, and Yang & Anderson, 38 THERIOGENOLOGY 315-335 (1992).

For the purposes of the present invention, the term “embryo” or “embryonic” as used herein includes a developing cell mass that has not implanted into the uterine membrane of a maternal host. Hence, the term “embryo” as used herein can refer to a fertilized oocyte, a cybrid, a pre-blastocyst stage developing cell mass, and/or any other developing cell mass that is at a stage of development prior to implantation into the uterine membrane of a maternal host. Embryos of the invention may not display a genital ridge. Hence, an “embryonic cell” is isolated from and/or has arisen from an embryo.

An embryo can represent multiple stages of cell development. For example, a one cell embryo can be referred to as a zygote, a solid spherical mass of cells resulting from a cleaved embryo can be referred to as a morula, and an embryo having a blastocoel can be referred to as a blastocyst.

The term “fetus” as used herein refers to a developing cell mass that has implanted into the uterine membrane of a maternal host. A fetus can include such defining features as a genital ridge, for example. A genital ridge is a feature easily identified by a person of ordinary skill in the art, and is a recognizable feature in fetuses of most animal species. The term “fetal cell” as used herein can refer to any cell isolated from and/or has arisen from a fetus or derived from a fetus. The term “non-fetal cell” is a cell that is not derived or isolated from a fetus.

The term “inner cell mass” as used herein refers to the cells that gives rise to the embryo proper. The cells that line the outside of a blastocyst are referred to as the trophoblast of the embryo. The methods for isolating inner cell mass cells from an embryo are well known to a person of ordinary skill in the art. See, Sims & First, 91 PROC. NATL. ACAD. SCI. USA 6143-47 (1994); and Keefer et al., 38 MOL. REPROD. DEV. 264-268 (1994). The term “pre-blastocyst” is well known in the art and is referred to previously.

A “transgenic embryo” refers to an embryo in which one or more cells contain heterologous nucleic acid introduced by way of human intervention. The transgene may be introduced into the cell, directly or indirectly, by introduction into a precursor of the cell, by way of deliberate genetic manipulation, or by infection with a recombinant virus. In the transgenic embryos described herein, the transgene causes cells to express a structural gene of interest. However, transgenic embryos in which the transgene is silent are also included.

The term “transgenic cell” refers to a cell containing a transgene.

The term “germ cell line transgenic animal” refers to a transgenic animal in which the genetic alteration or genetic information was introduced into a germ line cell, thereby conferring the ability to transfer the genetic information to offspring. If such offspring in fact possess some or all of that alteration of genetic information, they are transgenic animals as well.

The term “gene” refers to a DNA sequence that comprises control and coding sequences necessary for the production of a polypeptide or precursor. The polypeptide can be encoded by a full length coding sequence or by any portion of the coding sequence so long as the desired enzymatic activity is retained.

The term “transgene” broadly refers to any nucleic acid that is introduced into the genome of an animal, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form. The transgene may be specifically targeted to a defined genetic locus, may be randomly integrated within a chromosome, or it may be extrachromosomally replicating DNA. A transgene may include one or more transcriptional regulatory sequences and any other nucleic acid, such as introns, that may be necessary for optimal expression of a selected nucleic acid. A transgene can be coding or non-coding sequences, or a combination thereof. A transgene may comprise a regulatory element that is capable of driving the expression of one or more transgenes under appropriate conditions.

The phrase “a structural gene of interest” refers to a structural gene which expresses a biologically active protein of interest or an antisense RNA, for example. The structural gene may be derived in whole or in part from any source known to the art, including a plant, a fungus, an animal, a bacterial genome or episome, eukaryotic, nuclear or plasmid DNA, cDNA, viral DNA, or chemically synthesized DNA. The structural gene sequence may encode a polypeptide, for example, a receptor, enzyme, cytokine, hormone, growth factor, immunoglobulin, cell cycle protein, cell signaling protein, membrane protein, cytoskeletal protein, or reporter protein (e.g., green fluorescent proetin (GFP), β-galactosidase, luciferase). In addition, the structural gene may be a gene linked to specific disease or disorder such as a cardiovascular disease, neurological disease, reproductive disorder, cancer, eye disease, endocrine disorder, pulmonary disease, metabolic disorder, autoimmune disorder, and aging.

A structural gene may contain one or more modifications in either the coding or the untranslated regions which could affect the biological activity or the chemical structure of the expression product, the rate of expression, or the manner of expression control. Such modifications include, but are not limited to, mutations, insertions, deletions, and substitutions of one or more nucleotides. The structural gene may constitute an uninterrupted coding sequence or it may include one or more introns, bound by the appropriate splice junctions. The structural gene may also encode a fusion protein.

Primates, identical in both nuclear and cytoplasmic components, cannot be produced by current cloning strategies, yet these identicals represent ideal scientific models, for example, for preclinical investigations on the genetic and epigenetic basis of diseases. Here, the present invention relates to producing genetically identical primates as twin and higher-order multiples by the separation and reconstruction of blastomeres of cleavage-stage embryos, and pregnancies and birth results after embryo transfers. A total of 368 multiples have been created by splitting 107 rhesus embryos. Four pregnancies were established after the transfer of 13 split embryos (31% versus 53% controls). A healthy female was born from a quarter of an embryo, which demonstrates that this approach can result in live offspring. Her sibling, identical by DNA fingerprinting, aborted as a “blighted” pregnancy, i.e., normal placenta lacking fetal tissues. Blastocyst cell numbers were lower in multiples versus controls, and compaction and blastocyst formation occurred faster. Apoptosis occurred at higher rates in the inner cell mass (ICM) from split embryos; the resultant paucity of ICM cells may account for the blighted pregnancy. Blastomere biopsies may be performed in which a cell or two may be stored for possible stem cell therapy or genetic analysis (e.g., preimplantation genetic analysis), with the majority of the embryo implanted for procreation. Each of the split embryos may be frozen separately and stored, and eventually all of the embryos may be thawed and transferred successfully. Consequently, it is possible to produce identical offspring, with, for example, the same gestational mother in sequential pregnancies, so that the influences of fetal-maternal environments may be distinguished from both fetal and maternal genetics. Furthermore, the full potential of primate stem cells may be investigated using lines established from split embryos introduced into the genetically identical offspring. Cloning by splitting, instead of nuclear transfer, addresses the urgent requirements for primate research models that are both genetically identical and biologically normal. Thus, split embryos may be stored for subsequent pregnancies or in which stem cell lines, identical to a living offspring, may be tested for cell therapeutic potentials.

This cloning technology not only provides the means to produce genetically identical primates, but also the potential to produce genetically identical transgenic primates. These transgenic primates may be utilized as models for both the study of serious human diseases and for assessing the efficacy of gene and cell therapeutic strategies, thereby filling the scientific void between knock-out mice and human patients. The most favorable approaches for producing transgenic animals use modified donor cells either for nuclear transfer or for stem cell technologies. Since the former strategy is encountering seemingly insurmountable hurdles, the latter might prove feasible, but only if primate offspring can be produced from chimeric embryos using genetically engineered embryonic stem cells. Importantly, the present invention describes the success in primate embryo dissociation, manipulation, transfer to donor zonae, growth of reconstructed embryos, embryo transfer, the establishment of pregnancies, and the birth of offspring derived from a portion of an embryo: all steps for perfecting research protocols to establish the totipotency of stem cells and other chimeras in primates.

The failure of the blighted pregnancy raises the possibility of placental therapy, since these cells contributed to a functional placenta after implantation. Placental insufficiency leads to intrauterine fetal growth retardation, and therapy might utilize placental cell supplementation. Research potentials include propagation of embryos lost due to genomic imprinting (Gerard et al. (1999); Clark et al. (1999); Hiby et al. (1999) and Williamson et al., 72 GENET. RES. 255-65 (1998)), like androgenotes, and perhaps even the clones produced by nuclear transfer, if the primary etiology is indeed placental insufficiency (Cibelli et al. (1998); Hill et al. (1999); Renard et al. (1999); Wells et al. (1998); and Wells et al. (1999)). These donated cells could be tagged to ensure that they do not contribute to the ICM or fetus.

Implications for preimplantation genetic diagnosis include concerns about the accuracy after blastomere biopsies in light of the apoptosis rates, and also fetal viability after blastomere removal. Thus, it may be prudent to perform a genetic analysis on a blastomere isolated from an embryo prior to implantation. In addition to fetal viability, this analysis may be used to assess the integrity of chromosomal DNA, the presence of a transgene, and genetic mutations.

Numerous methods may be used for preimplantation genetic diagnosis. For example, PCR methods may be utilized for gene mutation analysis (Tsai, 19 PRENAT. DIAGN. 1048-51 (1999); Rojas et al. 64 FERTIL. STERIL. 255-60 (1995)). Multiplex marker PCR and multipex fluorescent PCR may be implemented to detect multiple mutations in a single cell (Dreesen et al., 6 MOL. HUM. REPROD. 391-96 (2000); Blake et al., 5 MOL. HUM. REPROD. 1166-75 (1999)). Another strategy for detection of multiple mutations is DGGE analysis (Vrettou et al., 19 PRENAT. DIAGN. 1209-16 (1999)). Other methods that may be used to detect genetic mutations include SSCP, heteroduplex analysis, and RFLP (Tawata et al., 12 GENET. ANAL. 125-27 (1996); Diamond et al., 27 BIOTECHNIQUES 1054-62 (1999); Van den Veyver and Roa, 10 CURR. OPIN. OBSTET. GYNECOL. 97-103 (1998); Sutterlin et al., 19 PRENAT. DIAGN. 1231-36 (1999)).

In addition, the single cell gel electrophoresis assay (COMET) may be used to assess DNA double- and single-strand breaks (Rojas et al., 722 J. CHROMATOGR. B. BIOMED. SCI. APPL. 225-54 (1999); Takahashi et al., 54 THERIOGENOLOGY 137-45 (2000); Takahashi et al., 54 MOL. REPROD. DEV. 1-7 (1999)). To detect chromosomal abnormalities, a FISH analysis may be performed (Sasabe et al., 16 J. ASSIST. REPROD. GENET. 92-96 (1999)); however, the PRINS method may be used as an alternative to in situ hybridization (Pellestor et al., 2 MOL. HUM. REPROD. 135-38 (1996)) and chromosomal aneuploidy may be detected by the CGH method (Voullaire et al., 19 PRENAT. DIAGN. 846-51 (1999)).

The present invention also relates to the storage of embryonic cells for the purpose of “cellular insurance,” i.e., the maintenance of frozen blastomeres as an embryonic stem cell repository. Indeed, blastocysts from, for example, quintuplets to octuplets may be used for establishing embryonic stem cells. These cell lines might prove invaluable for cell therapy, and the clinical issue may be raised as to whether a single blastomere beyond the 4-cell stage should be cryopreserved, as insurance against devastating diseases or other maladies or traumas.

In summary, cloning by embryo splitting produces identical embryos efficiently and results in the live birth of primate offspring. Splitting may result in identical offspring as well as the establishment of stem cell lines identical to born offspring. Indeed, frozen embryos may be stored for subsequent implantation and/or stem cell lines created for cell therapy.

While, in a particular embodiment of the present invention, primate quadruplets are the result of embryo splitting, sets of identical twin, triplet, quadruplet (or greater) primates are contemplated and enabled, and would permit, for example, such essential preclinical investigations.

Genetically identical cells and stem cells derived from primates may be invaluable for the study of numerous diseases (e.g., aging, AIDS, cancer, Alzheimer's disease, autoimmune diseases, metabolic disorders, obesity, organogenesis, psychiatric illnesses, and reproduction). Furthermore, the importance of these cells for molecular medicine and the development of innovative strategies for gene therapy protocols should not be minimized. For example, clinical strategies may include cloning, assisted reproductive technologies, transgenesis, and use of totipotent and immortalized embryonic germ (EG) and stem cells (ES). In addition, identical, transgenic and/or immortalized, totipotent EG- or ES-derived cells may be ideal preclinical models in identifying the molecular events related to infertility, gametogenesis, contraception, assisted reproduction, the genetic basis of infertility, male versus female meiotic cell cycle regulation, reproductive aging, and the non-endocrine basis of idiopathic infertility.

These technologies may also be utilized to study human development, particularly pre- and post-implantation development, body axis specification, somitogenesis, organogenesis, imprinting, extra-embryonic membrane allocation, and pluripotency. Using dynamic noninvasive imaging of transgenic reporters, the cell allocation in the primate fetus may be identified throughout pregnancy and life. Cloning and transgenesis may also be used to discover disease mechanisms and to create and optimize molecular medical cures. For example, primates created with a genetic knockout for a specific gene may accelerate discovery of the cures for cancer, arteriosclerosis causing heart disease and strokes, inborn errors of metabolism and other fetal and neonatal diseases, Parkinson's disease, polycystic kidney disease, blindness, deafness, sensory disorders, storage diseases (Lesch-Nyan and Zellwegers), and cystic fibrosis. These animals may also be amenable for evaluating and improving cell therapies including diabetes, liver damage, kidney disease, artificial organ development, wound healing, damage from heart attacks, brain damage following strokes, spinal cord injuries, memory loss, Alzheimer's disease and other dementia, muscle and nerve damage.

Thus, the present invention also relates to methods of using embryonic stem cells and transgenic embryonic stem cells to treat human diseases. Specifically, the methods for clonal propagation of primates, described in the present invention, may also be used to create embryonic stem cells and transgenic embryonic stem cells.

Cells from the inner cell mass of an embryo (i.e., blastocyst) may be used to derive an embryonic stem cell line, and these cells may be maintained in tissue culture (see, e.g., Schuldiner et al., 97 PROC. NATL. ACAD. SCI. USA 11307-12 (2000); Amit et al., 15 DEV. BIOL. 271-78 (2000); U.S. Pat. Nos. 5,843,780; 5,874,301 which are expressly incorporated by reference). In general, stems cells are relatively undifferentiated, but may give rise to differentiated, functional cells. For example, hemopoietic stem cells may give rise to terminally differentiated blood cells such as erythrocytes and leukocytes.

Using the methods described in the present invention, transgenic primate embryonic stem cells may also be produced which express a gene related to a particular disease. For example, transgenic primate embryonic cells may be engineered to express tyrosine hydroxylase which is an enzyme involved in the biosynthetic pathway of dopamine. In Parkinson's disease, this neurotransmitter is depleted in the basal ganglia region of the brain. Thus, transgenic primate embryonic cells expressing tyrosine hydroxylase may be grafted into the region of the basal ganglia of a patient suffering from Parkinson's disease and potentially restore the neural levels of dopamine (see, e.g., Bankiewicz et al., 144 EXP. NEUROL. 147-56 (1997)). The methods described in the present invention, therefore, may be used to treat numerous human diseases (see, e.g., Rathjen et al., 10 REPROD. FERTIL. DEV. 31-47 (1998); Guan et al., 16 ALTEX 135-41 (1999); Rovira et al., 96 BLOOD 4111-117 (2000); Muller et al., 14 FASEB J. 2540-48 (2000)).

### EXAMPLES

The present invention is further illustrated by the following examples which should not be construed as limiting in any way. The contents of all cited references (including literature references, issued patents, published patent applications, and co-pending patent applications) cited throughout this application are hereby expressly incorporated by reference.

### Example 1

### Embryo Splitting

Rhesus oocytes recovered by laparoscopy from gonadotropin stimulated female rhesus monkeys were fertilized by in vitro fertilization (IVF) (Wu et al., 55 BIOL. REPROD. 260-70 (1996)). Embryos were cultured until the appropriate stage and the zonas removed using pronase (Hewitson et al., 13 HUM. REPROD. 3449-55 (1998)). Zona-free embryos were allowed to recover individually for 20 minutes before splitting. Individual embryos were transferred into a manipulation drop containing calcium and magnesium-free TALP-HEPES medium. Blastomeres were dissociated by repeated aspiration through a blunt micropipet (I.D. 30 μm) controlled by a microsyringe. Dissociated blastomeres were transferred into an empty zona produced by mechanical removal of oocyte cytoplasm after zona splitting. Each multiple embryo produced was placed in its own zona to ensure blastomere aggregation. Consequently, zonae were limiting since there is only one zona per egg collected. To remedy this, additional zonae recovered from bovine oocytes were used successfully.

Surrogate females for embryo transfer were selected on the basis of serum estradiol and progesterone levels. Pregnancies were ascertained by endocrinological profiles and fetal ultrasound performed between days 24-30.

Parentage assignments were performed by DNA typing for 13 microsatellite loci amplified by polymerase chain reaction (PCR) with heterologous human primers for loci D3S1768, D6S276, D6S291, D6S1691, D7S513, D7S794, D8S1106, D10S1412, D11S925, D13S765, D16S403, D17S804, and D18S72.

Follicle Stimulation Regimen.

Hyperstimulation of female rhesus monkeys exhibiting regular menstrual cycles was induced with exogenous gonadotropins (Meng et al., 57 BIOL. REPROD. 454-59 (1997); Vandervoort et al., 6 J. IN VITRO FERTIL. EMBRYO TRANSFER 85-91 (1989); Zelinski-Wooten et al., 51 HUM. REPROD. 433-40 (19950). Beginning at menses, females were down-regulated by daily subcutaneous injections of a GnRH antagonist (Antide; Ares Serono, Aubonne, Switzerland; 0.5 mg/kg body weight) for 6 days during which recombinant human FSH (r-hFSH; Organon Inc., West Orange, N.J.; 30 IU, i.m.) was administered twice daily. This was followed by 1, 2, or 3 days of

r-hFSH plus r-hLH (r-hLH; Ares Serono; 30 IU each, i.m., twice daily). Ultrasonography was performed on day 7 of the follicle stimulation to confirm adequate follicular response. When follicles reached 3-4 mm in diameter, an i.m. injection of 1000 IU r-hCG (Serono, Randolph, Mass.) was administered for ovulation.

Follicular Aspiration by Laparoscopy:

Follicular aspiration was performed 27 hours post-hCG. Oocytes were aspirated from follicles using a needle suction device lined with Teflon tubing (Renou et al., 35 FERTIL. STERIL. 409-12 (1981) and modified by Bavister et al., 28 BIOL. REPROD. 983-99 (1993)). Briefly, a 10 mm trocar was placed through the abdominal wall and a telescope was introduced. Ovaries were visualized by a monitor attached to the inserted telescope. Two small skin incisions facilitate the insertion of 5 mm trocars bilaterally. Grasping forceps were introduced through each trocar to fixate the ovary at two points. Once stabilized, a 20-gauge stainless steel hypodermic needle with teflon tubing was attached to a OHMEDA vacuum regulator. The tubing was first flushed with sterile TALP-HEPES, supplemented with 5 IU/ml heparin and then inserted through the abdominal wall and into each ovary. Multiple individual follicles were aspirated with continuous vacuum at approximately 40-60 mm Hg pressure into blood collection tubes containing 1 ml of TALP-HEPES medium supplemented with 5 IU/ml heparin and maintained at 37° C. Collection tubes were immediately transported to a dedicated primate oocyte/zygote laboratory for oocyte recovery and evaluation of the maturation stage.

Collection and Evaluation of Rhesus Oocytes.

The contents of each collection tube was diluted in TALP-HEPES supplemented with 2 mg/ml hyaluronidase. Oocytes were rinsed and then transferred to pre-equilibrated CMRL medium containing 3 mg/ml BSA (CMRL-BSA) and supplemented with 10 mg/ml porcine FSH and 10 IU/ml hCG, prior to evaluation of maturational state. Metaphase II-arrested oocytes, exhibiting expanded cumulus cells, a distinct perivitelline space, and first polar body, were maintained in CMRL-BSA for up to 8 hours before fertilization. Immature oocytes were matured in CMRL-BSA plus hormones for up to 24 hours (Bavister et al. (1983); BOATMAN, IN VITRO GROWTH OF NON-HUMAN PRIMATE PRE- AND PERI-IMPLANTATION EMBRYOS 273-308 (B. D. Bavister, ed., Plenum Press 1987); Morgan et al., 45 BIOL. REPROD. 89-93 (19910).

Collection, Preparation, and Handling of Rhesus Sperm.

Rhesus males of proven fertility have been trained to routinely produce acceptable semen samples by penile electroejaculation (Bavister et al. (1983); Boatman (19870). After liquefaction of the coagulated ejaculate, the liquid semen was removed and washed three times in 10 ml of TALP-HEPES by centrifugation at 400× g for 5 minutes. Following resuspension of the pellet in 1 ml TALP-HEPES, a small sample was removed for structural analysis. The remainder was counted, diluted to a concentration of 20×10⁶ sperm/ml in 1 ml equilibrated TALP, and then placed in a 35 mm plastic Petri dish overlaid with 10 ml of mineral oil. Sperm suspensions were incubated at 37° C. under 5% CO₂ in air for 6 hours. Caffeine (1 mM) and 1 mM dibutyryl cyclic adenosine monophosphate (dbcAMP), were added for the final hour to stimulate hyperactivation (Bavister et al. (1983)). Sperm was used to perform IVF (Wu et al., 55 BIOL. REPROD. 260-70, 1996) and intracytoplasmic sperm injection (ICSI) (Hewitson et al., 55 BIOL. REPROD. 271-80 (1996)) for the generation of embryos. Blastomeres from cleavage stage embryos were dissociated and used as nuclear donors for nuclear transfer and fusion.

Embryo Splitting.

Splitting of embryos to produce genetically identical twins was accomplished by blastomere aspiration based on the methods described by Krzyminska et al. (5 HUMAN REPROD. 203-08 (1990)) for embryo biopsy. Four- to 8-cell IVF or ICSI embryos were transferred to 100 ml Ca²⁺ and Mg²⁺-free medium under oil and incubated for 10 minutes. An embryo was held by suction with the aid of a micropipette. A biopsy pipette (I.D. 30-40 mM) was introduced through the zona and the blastomeres were gently removed by aspiration. Alternatively, a blunt, flame polished micropipette was introduced through a hole in the zona (achieved using a fine stream of acid Tyrode's solution; Handyside et al., 1 LANCET 347-49 (1989)) and the blastomeres were removed by aspiration. The blastomeres were then inserted into empty zonae with the aid of micropipettes. Two twin embryos (one in the original zona, the other in an artificial zona) were washed twice in TALP-HEPES, once in CMRL, and then co-cultured in CMRL medium on BRL cells until cleavage occurred. The twin embryos were then used for transfer to surrogate females.

Selection of Recipients for Embryo Transfer.

Rhesus females with normal menstrual cycles synchronous with the egg donor were screened as potential embryo recipients. Screening was performed by collecting daily blood samples beginning on day 8 of the menstrual cycle (day 1 is the first day of menses) and analyzed for serum progesterone and estrogen. When serum estrogen levels increase to 2-4 times base level, ovulation usually follows within 12 to 24 hours. Timing of ovulation was detected by a significant decrease in serum estrogen levels and an increase in serum progesterone levels (e.g., to above 1 ng/ml). Surgical embryo transfers were performed on day 2 or 3 following ovulation by transferring two 4- to 8-cell embryos into the oviduct of the recipient.

Embryo Transfer by Laparotomy and Pregnancy Monitoring.

Surgical embryo transfers were performed by mid-ventral laparotomy (Wolf et al., 41 BIOL. REPROD. 335-46 (1989)). The oviduct was cannulated using a Tomcat catheter containing two 4- to 8-cell stage embryos in HEPES-buffered TALP, containing 3 mg/ml BSA. Embryos were expelled from the catheter in 0.05 ml of medium while the catheter was withdrawn. The catheter was flushed with medium following removal from the female to ensure that the embryos were successfully transferred. To confirm implantation, blood samples were collected daily and analyzed for serum estrogen and progesterone levels (Lanzendorf et al., 42 BIOL. REPROD. 703-11 (1990)). If hormone levels indicated a possible pregnancy, this was confirmed by transabdominal ultrasound on day 35 post-transfer. During the ultrasound, measurements were taken of total fetal length, fetal cardiac activity, and size of yolk sac. These measurements were compared to similar measurements gathered from IVF and natural pregnancies (Tarantal and Hendrickx, 15 AM. J. PRIMAT. 309-23 (1988)). Following confirmation of a pregnancy, blood samples were taken twice a week and monitored for serum progesterone and estrogen levels through the second trimester. Ultrasound was performed during the second trimester to determine developmental normalcy. In recipients with adequate estrogen and progesterone levels, but not pregnant based on ultrasound examination, blood samples were analyzed for serum monkey chorionic gonadotropin (mCG) measured by an LH.bioassay (Ellinwood et al., 22 BIOL. REPROD. 955-963 (1980)).

Detection of Apoptotic cells.

A terminal deoxynucleotidyl transferase (TdT) mediated dUTP nick-end labeling (TUNEL) assay kit (In Situ Death Detection Kit, Boehringer Mannheim, USA) was used to assess the presence of apoptotic cells. The complete fixation and TUNEL assay was performed in Terasaki dishes. Zona pellucida-free blastocysts were fixed in 2% formaldehyde (pH 7.4) for 30 minutes, rinsed in PBS, then permeabilized in PBS with 0.1% Triton X-100 and 0.1% NaCitrate solution at 4° C. for 2 minutes. The broken DNA ends of the embryonic cells were labeled with TdT and fluorescein-dUTP for 60 minutes at 37° C. The blastocyst were counter-stained with 1 μg/ml Hoechst 33258 (bisbenzimide trihydrochloride, Sigma, St Louis, Mo.) to visualize total DNA. The blastocysts were mounted onto glass slides using Vectashield (Vector Labs, CA). To prevent pressure on the blastocysts and to retain their three-dimensional structure, two coverglass spacers (170 μm height, i.e., >130-150 μm rhesus embryo diameters) were placed beneath the coverslip alongside the droplet of Vectorshield. Confocal image slices, serially spaced 3 μm apart, were collected with a Leica confocal TCS SP microscope equipped with a argon laser for UV and a second argon-488 laser for fluorescein excitation. A 25× objective with a 0.75 N.A. was used. Between 30-50 images per blastocyst were created. These slices were compiled to generate a 3-dimensional image of the blastocyst. Individual confocal images were analyzed using Adobe Photoshop (Adobe Systems, Mountain View, Calif.). The slices were stacked on top of each other to create a complete three-dimensional reconstruction of each imaged blastocyst. This three-dimensional reconstruction provided the total cell number by counting the nuclei slice by slice. By focusing on the slices in the middle of the blastocyst, one can distinguish between the TE and ICM nuclei. In these slices, the TE cells formed a ring one cell layer thick around the periphery of the blastocyst, while the ICM cells comprise a thicker accumulation of cells in the blastocoel cavity. Also, the ICM nuclei are in close proximity to each other. Furthermore, the ICM cells are not visible in the upper and lower slices. Stacking the slices obtained with the argon-krypton laser (TUNEL staining) and the UV laser (Hoechst, total DNA), was used to distinguish which nuclei had undergone apoptosis and whether these nuclei were TE or ICM cells.

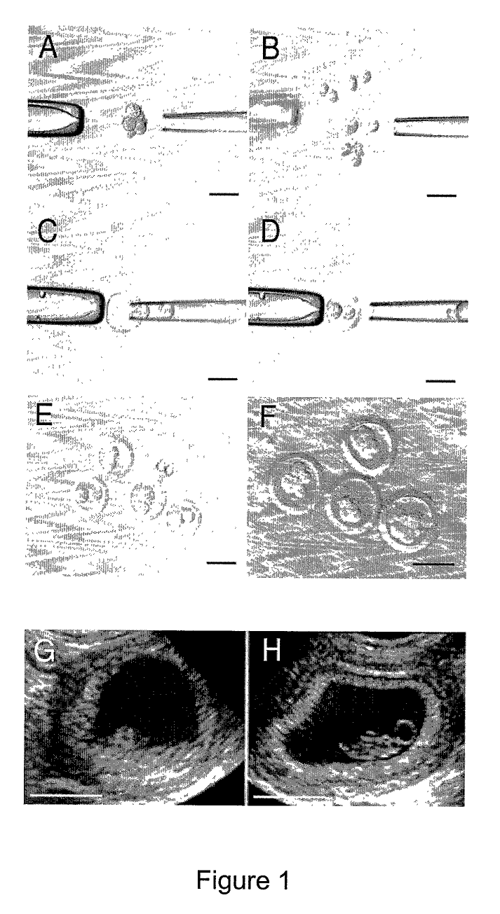

A total of 107 rhesus embryos were split to create 368 multiples. In FIG. 1A, an 8-cell embryo was split to produce a set of identical quadruplet embryos each comprised of two blastomeres. The zona-free, 8-cell embryo was dissociated into individual blastomeres (FIG. 1B). Each blastomere was handled by micromanipulation (FIG. 1C), and two blastomeres were inserted into an empty zona pellucida (FIG. 1D) creating one set of quadruplets (FIG. 1E) which were cultured in vitro (FIG. 1F). After transfer of a pair of the quadruplet embryos into two surrogates, proven as fertile breeders, both surrogates became pregnant. One surrogate (FIG. 1G) was identified on ultrasound as gestating a “blighted” pregnancy, i.e., a placental sac devoid of fetal tissue. Pedigree analysis by microsatellite based PCR demonstrates that it was genetically identical to the healthy female.

The healthy quadruplet female, was born at 157 days after an uneventful pregnancy (Hewitson et al., 5 NATURE MED. 431-33 (1999); Tarantal et al., 15 AM. J. PRIMAT. 309 (1988)). The initiation of pregnancy after embryo splitting and transfer into surrogates occurred at a frequency of 31% (4/13 versus 53.3% in controls) resulting in one biochemical pregnancy after transferring twin embryos (miscarried before thirty days of gestation); one biochemical quadruple pregnancy (FIG. 1G); and one live quadruple offspring (FIG. 1H). A fourth surrogate implanted with a twin embryo showed elevated chorionic gonadotropin levels. Four pregnancies (31%), but only one fetal sac and one live birth (8%) resulted from the thirteen transfers of multiple embryos. In contrast eight pregnancies (53%), ten fetal sacs (66%; due to twins) and six live births (40%) occurred in controls. Notwithstanding implantation evidence, factors accounting for the high pregnancy losses may include: the “donated” ruptured zona (though zona “drilling” is used clinically to improve implantation rates); the micromanipulation steps (though ICSI embryos develop at high rates after direct sperm microinjection); damage induced during blastomere dissociation; rhesus seasonality; and perhaps most likely, the fewer cells in the smaller multiple embryos.

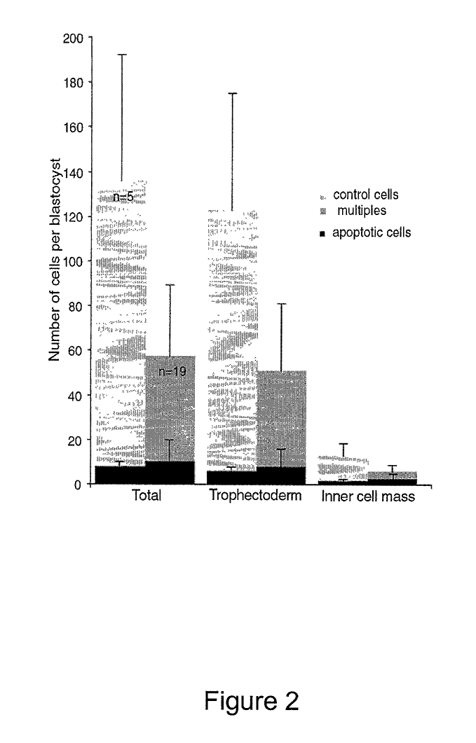

Blastocyst cell allocation was different in splits as compared to controls (FIG. 2). Embryonic cells have one of two fates: trophextoderm (TE; extraembryonic membrane precursors), or inner mass cell (ICM; fetal and extraembryonic membranes). Confocal imaging and 3-dimensional reconstruction of blastocytes from splits showed 6±2.6 ICM and 51.2±30.0 TE versus 13.2±4.8 ICM and 122.6±52.1 TE cells in IVF blastocysts (FIG. 2). Remarkably, primate blastocysts displayed bilateral symmetry, like mice, suggesting that the first meiotic axis specifies the embyronic plane separating the ICM from the blastoceol, and perhaps also the plane for gastrulation.

This reduction in ICM and TE cell number resulted in fewer progenitor cells and may therefore affect implantation rates and fetal development. The TUNEL assay determined that apoptosis is proportionally higher in the multiple embryos, and highest in the ICM cells of the multiples (39.9±35.3% versus 13.2±7.7% in controls). This may have contributed to the miscarriages, since TE cells have the capacity to implant, but too few ICM cells reduces viable fetal production.

Pregnancies were established with quadruplet embryos, and septuplet embryos retained the capacity to form blastocysts in vitro with viable ICM cells. In total, 59% of the multiple embryos underwent compaction, whereas only 12% of multiples retained the capacity to form a blastocyst. Most embryos were split at 40-48 hours post-insemination, ranging from the ₂^{nd} to ₄^{th} division, i.e., 4-16 cell embryos. The results of preimplantation development are shown in Table 1.

TABLE 1
Preimplantation Development

| | Number of cell division | | | | | | | | | |
|---|---|---|---|---|---|---|---|---|---|---|
| | 2 cells | | 3-4 cells | | 5-8 cells | | 9-16 cells | | 16-32 cells | |
| # of | (2^{nd)} | | (3^{rd)} | | (4^{th)} | | (5^{th)} | | (6^{th)} | |
| split | CM | Bl | CM | Bl | CM | Bl | CM | Bl | CM | Bl |
| 2 | 2/2 | 0/2 | 2/2 | 1/2 | 18/20 | 3/8 | 8/8 | 1/6 | | |
| 3 | | | 4/12 | 0/12 | 33/45 | 6/33 | 4/15 | 0/15 | | |
| 4 | | | 14/19 | 3/11 | 33/56 | 3/40 | 14/24 | 5/24 | | |
| 5 | | | | | 18/34 | 4/34 | 4/15 | 0/15 | | |
| 6 | | | | | 13/24 | 2/24 | | | | |
| 9 | | | | | | | | | 1/9 | 0/9 |
| total | 2/2 | 0/2 | 20/33 | 4/25 | 115/179 | 18/139 | 30/62 | 6/60 | 1/9 | 0/9 |

CM:Compaction; Bl: Blastocyst

Table 1: Preimplantation development in vitro of split embryos. Donor embryo stage, number of reconstructed identicals, and compacted morulae (CM) and blastocyst formation (BF) rates. Totals: <107 original embryos and <368 multiples since some have been frozen prior to compaction.

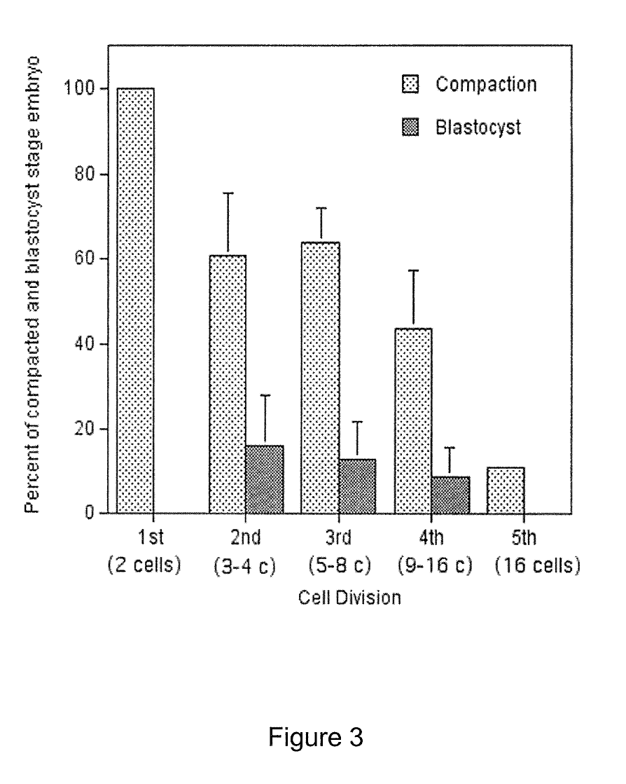

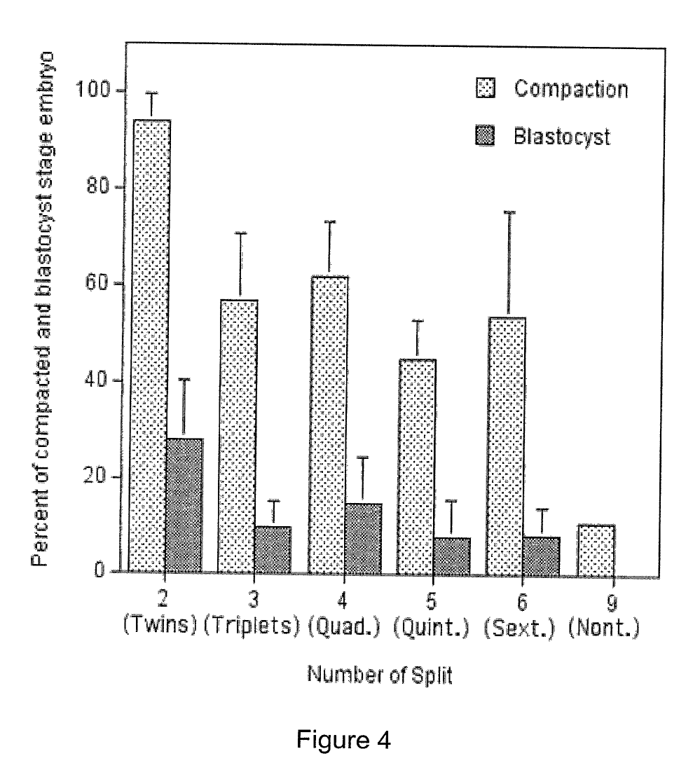

Compaction and blastocyst success rates declined at later stages (FIG. 3 and Table 1, supra). Also, the developmental potential of each individual reconstructed embryo decreased when higher order multiples were created from any single embryo (FIG. 4). When two embryos were reconstructed from an embryo, a high compaction rate (94%, n=32) with 28% blastocyst formation rate (n=18) was observed. Interestingly, reconstructed embryos compact slightly faster than controls, suggesting intrinsic chronological and/or cell-cycle clocks, rather than embryonic cell number. The molecular regulation of the maternal to embryonic transition, thought to occur in humans and other primates between the second and third divisions (i.e., 4-cell to 8-cell cleavages; Koford et al., 4 FOLIA PRIMATOL. 221-226 (1966); Braude et al., 322 NATURE 459-61 (1988)), corresponds to the loss of totipotency seen here in vitro as well as in nature. These cleavages may also specify cell fates as either the TE or the ICM (Fleming et al., 4 ANN. REV. CELL BIOL. 459-485 (1988)). Monozygotic twinning is rare naturally in mammals, e.g., 0.22% in rhesus, and <0.6% in humans (Benirschke, in ENCYCLOPEDIA OF REPRODUCTION, E. Knobil and J. D. Neill, Eds. (Academic Press, New York, 1999), vol. 4 pp. 887-891), except in some armadillos that always produce identical quadruplets by polyembryony. This exceptional example of asexual reproduction in mammals, i.e., the births of multiple offspring from a single fertilized egg, suggests that totipotency may be lost, at least in this species, at the 4-cell stage of development.

### Example 2

### Production of Embryonic Stem Cells

Embryonic stem (ES) cell are established from split embryos by the following method. Following embryo dissociation, 2-4 blastomeres are cultured in a microwell, which contains a monolayer of feeder cells derived from mouse embryonic fibroblasts (MEF). The remaining embryo is then transferred to an empty zona for embryo reconstruction as described in Example 1. This co-culture system for isolating and culturing an ES cell line is well known in the art (see, e.g., Thomson et al., 92 PROC. NATL. ACAD. SCI. USA 7844-48 (1995); Ouhibi et al., 40 MOL. REPROD. DEV. 311-24 (1995)). It has been suggested that the feeder cells provide growth factor-like leukemia inhibiting factor (LIF) which inhibits stem cell differentiation. The microwells contain 5-10 μl of culture medium (80% DMEM as a basal medium, 20% FBS, 1 mM β-mercaptoethanol, 1000 units/ml LIF, non-essential amino acids, and glutamine). The cells are then incubated at 37° C. with 5% CO₂ and covered with mineral oil. Fresh medium is replaced everyday and the survival of blastomeres is determined by cell division. During the initial culture, cell clumps are dissociated mechanically until cell attachment to the MEF monolayer and colony formation is observed. The colonies are then passaged to a 4-well plate and subsequently to a 35 mm dish in order to expand the culture gradually until a stable cell line is established. In addition to the dissociated blastomere culture, the reconstructed embryos are also cultured until the blastocyst stage is reached. Hatch blastocysts or blastocysts without zonae are cultured on a MEF monolayer in a microwell as described above. Instead of dissociating the blastomeres, the blastocysts are allowed to attach to the MEF monolayer. Once the blastocysts attach to the MEF, the ICM cells are isolated mechanically and transferred to a fresh culture well. The embryonic cells are cultured as described above and expansion of the cells is continued until individual colonies are observed. Individual colonies are selected for clonal expansion. This clonal selection and expansion process continues until a clonal cell line is established.

Infection of unfertilized oocytes by a pseudotyped retroviral vector has been used successfully to produce a transgenic nonhuman primate. These methods are disclosed in co-pending U.S. patent application Ser. No. 09/736,271, which is expressly incorporated herein by reference. The presence of the transgene was demonstrated in all tissues of the transgenic monkey, which suggests an early integration event has occurred, perhaps in the maternal chromosome prior to fertilization. To produce a transgenic embryonic stem cell line, the transgenic embryos produced by pseudotype infection are dissociated as described above in the clonal embryo production process. These split embryos are then used to produce clonal offspring or its embryonic counterpart is used to produce a transgenic embryonic stem cell line. Thus, the transgenic offspring and the transgenic embryonic stem cell line share the same genetic modification that was achieved at the oocyte stage.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

We claim:

1. A method for cloning an animal comprising the steps of:
dissociating blastomeres from embryos;
transferring said blastomeres to empty zonae;
culturing said blastomeres to an embryonic stage;
transferring said embryos to the oviducts of surrogate females; and
producing a cloned animal by parturition.

2. The method of claim 1, wherein said animal is selected from the group consisting of mammals, birds, reptiles, amphibians, and fish.

3. The method of claim 2, wherein said animal is a primate.

4. The method of claim 3, wherein said animal is a nonhuman primate.

5. The method of claim 4, wherein said nonhuman primate is a monkey.

6. The method of claim 1, wherein said embryo is at the 4- to 8-cell stage.

7. The method of claim 1, wherein said embryo is transgenic.

8. The method of claim 1, wherein said embryos are frozen and stored prior to said transferring to surrogate females.

9. The method of claim 1, further comprising the step of producing embryonic stem cells from said dissociated blastomeres.

10. The method of claim 7, further comprising the step of producing embryonic stem cells from said dissociated blastomeres.

11. An animal produced according to the method of claim 1.

12. The animal of claim 11, wherein said animal is a primate.

13. The animal of claim 12, wherein said animal is a nonhuman primate.

14. An animal produced according to the method of claim 7.

15. The animal of claim 14, wherein said animal is a primate.

16. The animal of claim 15, wherein said animal is a nonhuman primate.

17. A method of producing embryonic stem cells comprising the steps of:
dissociating blastomeres from embryos; and
culturing said blastomeres to produce stem cell lines.

18. The method of claim 17, wherein said embryonic stem cells are primate embryonic stem cells.

19. The method of claim 18, wherein said primate embryonic stem cells are nonhuman primate embryonic stem cells.

20. The method of claim 17, wherein said embryonic stem cells are transgenic embryonic stem cells.

21. The method of claim 20, wherein said transgenic embryonic stem cells are transgenic primate embryonic stem cells .

22. The method of claim 21, wherein said transgenic primate embryonic stem cells are transgenic nonhuman primate embryonic stem cells.

23. An embryonic stem cell produced according to the method of claim 17.

24. The embryonic stem cell of claim 23, wherein said embryonic stem cell is stored in an embryonic cell repository.

25. The embryonic stem cell of claim 23, wherein said embryonic stem cell is used for gene therapy.

26. The embryonic stem cell of claim 23, wherein said embryonic stem cell is used as a therapy for human disease.

27. The embryonic stem cell of claim 26, wherein said human disease is selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

28. An embryonic stem cell produced according to the method of claim 18.

29. The embryonic stem cell of claim 28, wherein said embryonic stem cell is stored in an embryonic cell repository.

30. The embryonic stem cell of claim 28, wherein said embryonic stem cell is used for gene therapy.

31. The embryonic stem cell of claim 28, wherein said embryonic stem cell is used as a therapy for human disease.

32. The embryonic stem cell of claim 31, wherein said human disease is selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

33. An embryonic stem cell produced according to the method of claim 19.

34. The embryonic stem cell of claim 33, wherein said embryonic stem cell is stored in an embryonic cell repository.

35. The embryonic stem cell of claim 33, wherein said embryonic stem cell is used for gene therapy.

36. The embryonic stem cell of claim 33, wherein said embryonic stem cell is used as a therapy for human disease.

37. The embryonic stem cell of claim 36, wherein said human disease is selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

38. An embryonic stem cell produced according to the method of claim 20.

39. The embryonic stem cell of claim 38, wherein said embryonic stem cell is stored in an embryonic cell repository.

40. The embryonic stem cell of claim 38, wherein said embryonic stem cell is used for gene therapy.

41. The embryonic stem cell of claim 38, wherein said embryonic stem cell is used as a therapy for human disease.

42. The embryonic stem cell of claim 41, wherein said human disease is selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

43. An embryonic stem cell produced according to the method of claim 21.

44. The embryonic stem cell of claim 43, wherein said embryonic stem cell is stored in an embryonic cell repository.

45. The embryonic stem cell of claim 43, wherein said embryonic stem cell is used for gene therapy.

46. The embryonic stem cell of claim 43, wherein said embryonic stem cell is used as a therapy for human disease.

47. The embryonic stem cell of claim 46, wherein said human disease is selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

48. An embryonic stem cell produced according to the method of claim 22.

49. The embryonic stem cell of claim 48, wherein said embryonic stem cell is stored in a repository.

50. The embryonic stem cell of claim 48, wherein said embryonic stem cell is used for gene therapy.

51. The embryonic stem cell of claim 48, wherein said embryonic stem cell is used as a therapy for human disease.

52. The embryonic stem cell of claim 51, wherein said human disease is selected from the group consisting of cardiovascular diseases, neurological diseases, reproductive disorders, cancers, eye diseases, endocrine disorders, pulmonary diseases, metabolic disorders, hereditary diseases, autoimmune disorders, and aging.

53. The method of claim 1, further comprising the step of performing preimplantation genetic diagnosis on said embryo.

54. The method of claim 53, wherein said preimplantation genetic diagnosis is performed prior to transfer to the oviduct of a female surrogate.

55. The method of claim 54, wherein said preimplantation genetic diagnosis is selected from the group comprising PCR, FISH, SSCP, RFLP, PRINS, CGH, COMET analysis, heteroduplex analysis, Southern analysis, and DGGE analysis.

56. A method for preimplantation genetic diagnosis of an embryo comprising the steps of:
dissociating a blastomere from an embryo; and
performing genetic analysis on said blastomere prior to implantation of said embryo.

57. The method of claim 56, wherein said embryo is implanted into a female surrogate.

58. The method of claim 56, wherein said genetic analysis is selected from the group comprising PCR, FISH, SSCP, RFLP, PRINS, CGH, COMET analysis, heteroduplex analysis, Southern analysis, and DGGE analysis.

59. The method of claim 1, wherein said blastomeres are frozen.

60. The method of claim 59, wherein said blastomeres are stored in a repository.
